(19) United States
(12) Patent Application Publication
(10) Pub. No.: US 2010/0056536 A1
(43) Pub. Date: Mar. 4, 2010

(54) METHOD OF TREATING ATRIAL FIBRILLATION

(76) Inventors: Charles Antzelevitch, New Hartford, NY (US); Alexander Burashnikov, New Hartford, NY (US); John Shryock, East Palo Alto, CA (US); Sridharan Rajamani, Fremont, CA (US); Luiz Belardinelli, Palo Alto, CA (US)

Correspondence Address: CV THERAPEUTICS, INC.;Gilead Palo Alto, Inc., 333 Lakeside Drive, Foster City, CA, 94404, US

(21) Appl. No.: 12/553,841

(22) Filed: Sep. 3, 2009

Related U.S. Application Data
(60) Provisional application No. 61/108,776, filed on Oct. 27, 2008.
(60) Provisional application No. 61/094,359, filed on Sep. 4, 2008.

(51) Int. Cl. A61K 31/495; A61P 9/00

(52) U.S. Cl. 514/252.12

(57) ABSTRACT

The present invention relates to a method for the treatment of atrial fibrillation comprising the coadministration of a synergistic therapeutically effective amount of amiodarone and synergistic therapeutically effective amount ranolazine. This invention also relates to pharmaceutical formulations that are suitable for such combined administration.

### CROSS-REFERENCE TO RELATED APPLICATIONS

This application claims priority to U.S. Provisional Patent Application No. 61/108,776, filed Oct. 27, 2008, and U.S. Provisional Patent Application No. 61/094,359, filed Sep. 4, 2008, the entirety of which are incorporated herein by reference.

### FIELD OF THE INVENTION

The present invention relates to method of treating atrial fibrillation by combined administration of therapeutically effective amounts ranolazine and amiodarone. The method finds utility in the treatment of arrhythmia, particularly atrial fibrillation. This invention also relates to pharmaceutical formulations that are suitable for such combined administration.

### DESCRIPTION OF THE ART

U.S. Pat. No. 4,567,264, the specification of which is incorporated herein by reference in its entirety, discloses ranolazine, (±)-N-(2,6-dimethylphenyl)-4-[2-hydroxy-3-(2-methoxyphenoxy)-propyl]-1-piperazineacetamide, and its pharmaceutically acceptable salts, and their use in the treatment of cardiovascular diseases, including arrhythmias, variant and exercise-induced angina, and myocardial infarction. In its dihydrochloride salt form, ranolazine is represented by the formula:

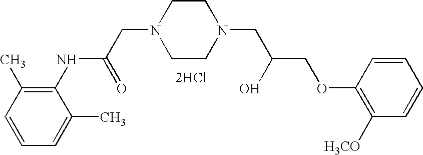

This patent also discloses intravenous (IV) formulations of dihydrochloride ranolazine further comprising propylene glycol, polyethylene glycol 400, Tween 80 and 0.9% saline.

U.S. Pat. No. 5,506,229, which is incorporated herein by reference in its entirety, discloses the use of ranolazine and its pharmaceutically acceptable salts and esters for the treatment of tissues experiencing a physical or chemical insult, including cardioplegia, hypoxic or reperfusion injury to cardiac or skeletal muscle or brain tissue, and for use in transplants. Oral and parenteral formulations are disclosed, including controlled release formulations. In particular, Example 7D of U.S. Pat. No. 5,506,229 describes a controlled release formulation in capsule form comprising microspheres of ranolazine and microcrystalline cellulose coated with release controlling polymers. This patent also discloses IV ranolazine formulations which at the low end comprise 5 mg ranolazine per milliliter of an IV solution containing about 5% by weight dextrose. And at the high end, there is disclosed an IV solution containing 200 mg ranolazine per milliliter of an IV solution containing about 4% by weight dextrose.

The presently preferred route of administration for ranolazine and its pharmaceutically acceptable salts and esters is oral. A typical oral dosage form is a compressed tablet, a hard gelatin capsule filled with a powder mix or granulate, or a soft gelatin capsule (softgel) filled with a solution or suspension. U.S. Pat. No. 5,472,707, the specification of which is incorporated herein by reference in its entirety, discloses a high-dose oral formulation employing supercooled liquid ranolazine as a fill solution for a hard gelatin capsule or softgel.

U.S. Pat. No. 6,503,911, the specification of which is incorporated herein by reference in its entirety, discloses sustained release formulations that overcome the problem of affording a satisfactory plasma level of ranolazine while the formulation travels through both an acidic environment in the stomach and a more basic environment through the intestine, and has proven to be very effective in providing the plasma levels that are necessary for the treatment of angina and other cardiovascular diseases.

U.S. Pat. No. 6,852,724, the specification of which is incorporated herein by reference in its entirety, discloses methods of treating cardiovascular diseases, including arrhythmias variant and exercise-induced angina and myocardial infarction.

U.S. Patent Application Publication Number 2006/0177502, the specification of which is incorporated herein by reference in its entirety, discloses oral sustained release dosage forms in which the ranolazine is present in 35-50%, preferably 40-45% ranolazine. In one embodiment the ranolazine sustained release formulations of the invention include a pH dependent binder; a pH independent binder; and one or more pharmaceutically acceptable excipients. Suitable pH dependent binders include, but are not limited to, a methacrylic acid copolymer, for example Eudragit® (Eudragit® L100-55, pseudolatex of Eudragit® L100-55, and the like) partially neutralized with a strong base, for example, sodium hydroxide, potassium hydroxide, or ammonium hydroxide, in a quantity sufficient to neutralize the methacrylic acid copolymer to an extent of about 1-20%, for example about 3-6%. Suitable pH independent binders include, but are not limited to, hydroxypropylmethylcellulose (HPMC), for example Methocel® E10M Premium CR grade HPMC or Methocel® E4M Premium HPMC. Suitable pharmaceutically acceptable excipients include magnesium stearate and microcrystalline cellulose (Avicel® pH101).

### BACKGROUND

Atrial fibrillation (AF) is the most prevalent arrhythmia, the incidence of which increases with age. It is estimated that 8% of all people over the age of 80 experiences this type of abnormal heart rhythm and it accounts for ⅓ of hospital admission for cardiac rhythm disturbances. Approximately 2.2 million people are believed to have AF in the Unites States alone. Fuster et al Circulation 2006 114 (7): e257-354. Although atrial fibrillation is often asymptomatic it may cause palpitations or chest pain. Prolonged atrial fibrillation often results in the development of congestive heart failure and/or stroke. Heart failure develops as the heart attempts to compensate for the reduced cardiac efficiency while stroke may occur when thrombi form in the atria, pass into the blood stream and lodge in the brain. Pulmonary emboli may also develop in this manner.

Current methods for treating AF include electric and/or chemical cardioversion and laser ablation. Anticoagulants such as warfarin and heparin are typically prescribed in order to avoid stroke. While there is currently some debate regarding the choice between rate and rhythm control, see Roy et al. N. Engl. J Med 2008 358: 25; 2667-2677, rate control is typically achieved by the use of beta blockers, cardiac glycosides, and calcium channel blockers.

One of the most comment anti-arrhythmic agents is amiodarone which is commonly administered for both acute and chronic arrhythmias including acute and/or chronic AF. Unfortunately amiodarone is a highly toxic drug and has a wide range of undesirable side effects. The most dangerous of these effects is the development of interstitial lung disease. Thyroid toxicity, both hypothyroidism and hyperthyroidism, is often seen as are effects in the eye and liver. Most if not all of these undesirable side effects are dose dependent and so methods of increasing the efficacy of amiodarone to enable a reduction of dose are highly desirable,

It has now been discovered that the combination of chronic amiodarone and relatively low concentrations of acute ranolazine produces a synergistic use-dependent depression of sodium channel-dependent parameters in isolated canine atria, leading to a potent effect of the drug combination to prevent the induction of atrial fibrillation.

### SUMMARY OF THE FIGURES

FIG. 1 presents the voltage dependence of activation and steady-state inactivation of sodium current in canine atrial versus ventricular myocytes. (A) Current-voltage relationship for sodium current in ventricular and atrial myocytes. Peak I_{Na} current density is larger in atrial versus ventricular myocytes. (B) Summarized steady-state inactivation curves. The half-inactivation voltage (V_{0.5}) is −88.80±0.19 mV in atrial cells (n=9) and −72.64±0.14 mV in ventricular cells (P<0.001, n=7). Insets show representative atrial and ventricular traces after 1-s conditioning pulses to the indicated potentials. (C) Steady-state inactivation curves before and after addition of 15 μM ranolazine. Ranolazine shifts V0.5 from −72.53±0.16 mV to −74.81±0.14 mV (P<0.01) in ventricular myocytes (n−4) and from −86.35±0.19 to −91.38±0.35 mV (P<0.001) in atrial myocytes (n=5).

FIG. 2 displays the atrial-selective suppression of the maximum rate of rise of the action potential upstroke (Vₘₐₓ) by ranolazine, lidocaine, and chronic amiodarone, but not propafenone in canine coronary artery—perfused atrial and ventricular preparations as discussed in Example 2. *P<0.05 versus respective control (C); †P<0.05 versus respective ventricular values, n=8-15. CL=500 ms.

FIG. 3 shows atrial selectivity of ranolazine in depressing Vₘₐₓ at fast pacing rates. Shown are action potential tracings and corresponding Vₘₐₓ values recorded during acceleration of pacing rate from a CL of 500 to 300 ms in atrial and ventricular preparations in the presence of Ranolazine as discussed in Example 2. Ranolazine prolongs repolarization of the AP in atria, but not in ventricles. Acceleration of rate leads to elimination of the diastolic interval in atria, resulting in a more positive take-off potential and a depression of Vₘₐₓ. The diastolic interval remains relatively long in ventricles.

### SUMMARY OF THE INVENTION

In one aspect of the invention a method is provided for the treatment of atrial fibrillation comprising the coadministration of a synergistic therapeutically effective amount of amiodarone and synergistic therapeutically effective amount of ranolazine. The two agents may be administered separately or together in separate or a combined dosage unit. If administered separately, the ranolazine may be administered before or after administration of the amiodarone but typically the ranolazine will be administered prior to the amiodarone.

In another aspect of the invention a method for reducing the undesirable side effects of amiodarone is presented. The method comprises coadministration of a synergistic therapeutically effective dose of amiodarone and a synergistic therapeutically effective dose of ranolazine. As before, the two agents may be administered separately or together in separate or a combined dosage unit. If administered separately, the ranolazine may be administered before or after administration of the amiodarone but typically the ranolazine will be administered prior to the amiodarone.

### DETAILED DISCRETION OF THE INVENTION

### Definitions and General Parameters

As used in the present specification, the following words and phrases are generally intended to have the meanings as set forth below, except to the extent that the context in which they are used indicates otherwise.

“Optional” or “optionally” means that the subsequently described event or circumstance may or may not occur, and that the description includes instances where said event or circumstance occurs and instances in which it does not.

The term “beta-blocker” refers to an agent that binds to a beta-adrenergic receptor and inhibits the effects of beta-adrenergic stimulation. Beta-blockers decrease AV nodal conduction. In addition, Beta-blockers decrease heart rate by blocking the effect of norepinephrine on the post synaptic SA nodal cells that control heart rate. Beta blockers also decrease intracellular Ca++ overload, which inhibits after-depolarization mediated automaticity. Examples of beta-blockers include, but are not limited to, acebutolol, atenolol, betaxolol, bisoprolol, carteolol, labetalol, metoprolol, nadolol, oxprenolol, penbutolol, pindolol, propranolol, sotalol, timolol, esmolol, sotalol, carvedilol, medroxalol, bucindolol, levobunolol, metipranolol, celiprolol, and propafenone.

“Parenteral administration” is the systemic delivery of the therapeutic agent via injection to the patient.

“Synergistic” means that the therapeutic effect of amiodarone when administered in combination with ranolazine (or vice-versa) is greater than the predicted additive therapeutic effects of amiodarone and ranolazine when administered alone.

The term “therapeutically effective amount” refers to that amount of a compound of Formula I that is sufficient to effect treatment, as defined below, when administered to a mammal in need of such treatment. The therapeutically effective amount will vary depending upon the specific activity of the therapeutic agent being used, the severity of the patient's disease state, and the age, physical condition, existence of other disease states, and nutritional status of the patient. Additionally, other medication the patient may be receiving will effect the determination of the therapeutically effective amount of the therapeutic agent to administer.

The term “treatment” or “treating” means any treatment of a disease in a mammal, including:
(i) preventing the disease, that is, causing the clinical symptoms of the disease not to develop;
(ii) inhibiting the disease, that is, arresting the development of clinical symptoms; and/or
(iii) relieving the disease, that is, causing the regression of clinical symptoms.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

### The Method of the Invention

The present invention relates to methods of treating or preventing atrial fibrillation. The method comprises coadministration of a synergistic therapeutically effective amount of amiodarone and synergistic therapeutically effective amount ranolazine. The two agents may be administered separately or together in separate or a combined dosage unit. If administered separately, the ranolazine may be administered before or after administration of the amiodarone but typically the ranolazine will be administered prior to the amiodarone

Ranolazine is an anti-ischemic and antianginal agent that has been shown in preclinical and clinical studies to inhibit the late sodium current (I_{Na}) and improve diastolic relaxation. In preclinical studies, ranolazine has also been shown to prevent cellular calcium overload and reduce cardiac electrical and mechanical dysfunction during ischemia.

Results of several recent studies have demonstrated that ranolazine reduces atrial arrhythmic activity. See Burashnikov et al. 2007;116: 1449-1457; Song et al. Am J Physiol 2008; 294: H2031-2039; Sicouri et al. Heart Rhythm 2008; 5: 1019-1026. Ranolazine was reported to cause greater inhibition of sodium channels in atrial than in ventricular tissue (Burashnikov et al. 2007;116: 1449-1457). Ranolazine at clinically relevant concentrations of 5 and 10 μM prolonged the duration of the action potential (APD₉₀, duration of the action potential at 90% of repolarization) in atria but had minimal or no effect on APD in ventricular myocardium (Burashnikov et al. 2007;116: 1449-1457). Ranolazine (5 and 10 μM) caused significant use-dependent (i.e., the effect of ranolazine was greater at higher rates of pacing) depression of the maximum rate of rise of the action potential upstroke [Vₘₐₓ] and conduction velocity in atrial myocardium and pulmonary vein sleeves but not in ventricular myocardium (Antzelevitch et al. Circulation 2004;110: 904-910, Burashnikov et al. Circulation 2007;116: 1449-1457, and Sicouri et al. Heart Rhythm 2008;5: 1019-1026). Ranolazine increased the effective refractory period, induced post-repolarization refractoriness, and caused a loss of excitability of the tissue at higher pacing rates in atrial tissue (Antzelevitch et al. Circulation 2004;110: 904-910, Burashnikov et al. Circulation 2007;116:1449-1457, Sicouri et al. Heart Rhythm 2008; 5:1019-1026) and Kumar et al. J Cardiovasc Electrophysiol 2009;20:796-802.

These data suggest that ranolazine would be effective to terminate and to reduce both the initiation and continuation of atrial tachycardia and fibrillation, and indeed ranolazine significantly depressed atrial excitability and both prevented and terminated acetylcholine-induced fibrillation in atrial myocardium and in canine pulmonary vein sleeves and porcine hearts. Burashnikov et al. 2007;116: 1449-1457, Sicouri et al. Heart Rhythm 2008; 5: 1019-1026, and and Kumar et al. J Cardiovasc Electrophysiol 2009;20:796-802 Ranolazine also abolished late I_{Na}-induced delayed afterdepolarizations and triggered activity of isolated atrial myocytes (Song et al. Am J Physiol 2008; 294: H2031-2039) and decreased diastolic depolarization and initiation of arrhythmic activity. Song et al. Am J Physiol 2009. in press.

Ranolazine appears to reduce both the triggers (delayed afterdepolarizations, excitability, and triggered activity) and the electrical substrate (atrial tissue that can support rapid conduction and a high rate of electrical activity) that initiate and support atrial tachycardia and fibrillation. Inhibition by ranolazine of specific ion channel currents (peak I_{Na}, I_{Kr}, and late I_{Na}) in atrial tissue is responsible for these anti-arrhythmic effects. First, atrial-selective reduction of peak I_{Na} by ranolazine reduces electrical impulse conduction (conduction velocity) and excitability. Second, inhibition by ranolazine of the delayed rectifier current I_{Kr} further slows the already slow terminal phase of repolarization of the atrial action potential and thereby reduces the availability of Na⁺ channels for activation of a subsequent action potential upstroke.

These effects contribute to a lengthening of the atrial effective refractory period and result in the induction of post-repolarization refractoriness of the tissue. Tissue that is refractory to electrical stimulation cannot support either the re-entry of electrical activity or high rates of stimulation such as those that occur during atrial tachycardia and fibrillation. Thus the effect of ranolazine to cause a rate-dependent increase of atrial refractoriness reduces the excitable substrate capable of supporting atrial fibrillation.

Finally, the reduction by ranolazine of late I_{Na} may contribute to reduction of cellular calcium loading and suppression of triggered activity in atria, particularly in the conditions of prolonged atrial repolarization, thus preventing the initiation of AF (Sicouri et al. Heart Rhythm 2008; 5:1019-1026; Song et al. 2008). Prolonged atrial APD may occur in a number of diseases associated with AF occurrence, such as the congestive heat failure (Li et al. Circulation 2000;101:2631-2638), atrial dilatation (Verheule at al. Circulation 2003; 107:2635-2622), hypertension (Kistler et al. Eur Heart J 2006;27:3045-3056), and long QT syndrome. (Kirchhof et al. J Cardiovasc, Electrophysiol 2003; 14:1027-1033).

However, AF is commonly associated with abbreviation of atrial repolarization. The integral of sodium ion influx is much smaller through late I_{Na} vs. early I_{Na} under normal conditions. With abbreviation of APD, this difference is expected to increase. As a consequence, specific inhibition of late I_{Na} may not significantly affect intracellular sodium concentration (compared to inhibition of early I_{Na}). Although ranolazine is a potent late I_{Na} blocker in the ventricle (Antzelevitch et al. Circulation 2004; 110: 904-910), its anti-AF actions in the canine right atria and pulmonary vein preparations are attributed primarily to its inhibition of early I_{Na} (Burashnikov et al. Circulation 2007;116:1449-1457 and Sicouri et al. Heart Rhythm 2008; 5: 1019-1026). In summary, strong evidence from preclinical studies suggests that ranolazine may be effective in suppressing atrial fibrillation in humans.

It has now been discovered that the concurrent use of ranolazine and low-dose amiodarone is a highly useful method to terminate and prevent atrial fibrillation. It is well known that amiodarone-induced thyroid toxicity may be reduced when the dose of the drug is reduced. The effect of acute amiodarone in low to moderate concentrations to cause Torsades de pointes can be explained by the action of the drug to inhibit I_{Kr} at lower concentrations than it inhibits late I_{Na} (Wu L et al., Cardiovasc Res 2008; 77: 481-488). Inhibition by amiodarone of I_{Kr} can increase the risk for development of Torsades. Ranolazine reduces late I_{Na} and has been shown to prevent Torsades de pointes caused by I_{Kr}-blocking drugs such as amiodarone (Wu L et al., JPET, 2006). Ranolazine has the potential to offset the inhibition of I_{Kr} and the consequent reduction of repolarization reserve caused by amiodarone, by reducing late I_{Na} and thereby increasing repolarization reserve. Because the pathological conditions in which late I_{Na} is reported to be enhanced are relatively common, the use of ranolazine to inhibit late I_{Na} before the administration of an I_{Kr} blocker such as amiodarone may be useful to reduce the incidence of ventricular tachyarrhythmias in patients.

The combination of ranolazine and amiodarone leads to strong inhibition of the sodium channels responsible for early (peak) I_{Na}. Whereas ranolazine is reported to be a Na+ channel “open and inactivated state” blocker with fast “off” kinetics (Wang et al. Mol Pharmacol 2008;73:940-948 and Zygmunt et al. Biophys J;2009:96:250a [abstract]), amiodarone is reported to be an “inactivated-state” blocker also with rapid kinetics (Kodama et al. Cardiovasc Res 1997;35:13-29). The combination of the two drugs results in an increased block of early I_{Na}. In the atria, both ranolazine and amiodarone inhibit I_{Kr} and therefore increase the atrial effective refractory period. The synergism of effects of ranolazine and amiodarone to increase the atrial diastolic threshold for excitation and to lengthen the effective atrial refractory period is expected to greatly reduce atrial excitability and therefore the frequency and duration of atrial tachycardias.

Recent studies have demonstrated that chronic amiodarone is an atrial-selective inactivated-state blocker of cardiac sodium channels and that ranolazine is an atrial-selective activated-state blocker of these channels (Wang et al. Mol Pharmacol 2008;73:940-948 and Zygmunt et al. Biophys J;2009:96:250a [abstract]) and have purposed atrial-selective sodium channel block as a strategy for suppression of atrial fibrillation. (Burashnikov et al. Heart Rhythm 2008; 5:1735-1742, Burashnikov et al. Ann N Y Acad Sci 2008; 1123:105-112; Burashnikov et al. Circ, 2007;116:1449-1457)

Ranolazine and amiodarone may be given to the patient in either single or multiple doses by any of the accepted modes of administration of agents having similar utilities, for example as described in those patents and patent applications incorporated by reference, including buccal, by intra-arterial injection, intravenously, intraperitoneally, parenterally, intramuscularly, subcutaneously, orally, or via an impregnated or coated device such as a stent, for example, or an artery-inserted cylindrical polymer.

When administered alone amiodarone is typically administered in a two stage processes. A loading dose is first given in order to achieve the therapeutic effect followed by a lower maintenance dose which sustains the therapeutic effect. When administered intravenously, the loading dose of amiodarone is recommended to be 150 mg over the first 10 minutes (15 mg/min) followed by 360 mg over the next 6 hours (1 mg/min). The maintenance infusion is then 540 mg over the remaining 18 hours of the first day of therapy (0.5 mg/min). The maintenance dose then continues for the remaining period of treatment at an infusion rate of 0.5 mg/min (720 mg/24 hours).

When ranolazine is coadministered with IV amiodarone the amiodarone loading dose duration may be decreased as may the amiodarone maintenance dose level. One of ordinary skill in the art will be able to ascertain the specific reduction in amiodarone loading time made possible by the coadministration of ranolazine as the treatment effect will be observed at an earlier time point than would normally be seen absent the ranolazine. In one embodiment the amiodarone loading dose is 150 mg for the first 10 minutes followed by 360 mg for the next two to four hours. The maintenance dose may then be decreased from the 720 mg/24 hours typically given to 540 mg, 360 mg, or 180 mg per day. As before, one of ordinary skill in the art will be able to ascertain the specific reduction in amiodarone dosage made possible by the coadministration of ranolazine as the treatment effect will be maintained at a lower dosage than would normally be possible absent ranolazine coadministration.

The IV ranolazine is administered in an IV solution comprising a selected concentration of ranolazine of from about 1.5 to about 3 mg per milliliter, preferably about 1.8 to about 2.2 mg per milliliter and, even more preferably, about 2 mg per milliliter. The infusion of the intravenous formulation of ranolazine is initiated such that a target peak ranolazine plasma concentration of about 2500 ng base/mL (wherein ng base/mL refers to ng of the free base of ranolazine/mL) is achieved and sustained.

Oral administration of amiodarone is also usually carried using loading and maintenance dosing. With oral administration, loading doses of 800 to 1,600 mg/day are typically required for 1 to 3 weeks (occasionally longer) until initial therapeutic response occurs. With coadministration of ranolazine initial loading doses (1200 to 1,600 mg/day) may be given for a shorter duration (7 to 10 days) before shifting to a smaller than typical maintenance dose, i.e., the maintenance dose may be reduced from the customary 400 mg per day to a much lower 200, 100, or 50 mg per day. Once again one of ordinary skill in the art will be able to ascertain the specific reduction in amiodarone dosage made possible by the coadministration of ranolazine as the treatment effect will be maintained at a lower dosage than would normally be possible absent ranolazine coadministration.

The reduction in the amiodarone maintenance dose is of particular advantage to those patents who are currently on oral amiodarone but are suffering from various adverse side effects of the drug. By adding ranolazine to the current therapy, the dosage of amiodarone may be substantially reduced as discussed above thereby alleviating amiodarone's more deleterious side effects.

In one embodiment then, the patient under treatment is already taking a maintenance dose of amiodarone ranging from 400 to 800 mg with a typical dose being 400 mg daily. To this dosing regimen is then added ranolazine at 1000 mg twice daily (2×500 mg), 750 mg twice daily (2×375 mg), 500 mg twice daily (1×500 mg), or 375 mg twice daily (1×375 mg). By administering such therapeutic doses of ranolazine the amount of amiodarone can then be decreased to 200, 100, or 50 mg daily thereby greatly reducing the incidence of adverse events.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present invention. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the component in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The ideal forms of the apparatus for administration of the novel combinations for atrial fibrillation, consist therefore of (1) either a syringe comprising 2 compartments containing the 2 active substances ready for use or (2) a kit containing two syringes ready for use.

In making a pharmaceutical compositions that include ranolazine and amiodarone, the active ingredients are usually diluted by an excipient and/or enclosed within such a carrier that can be in the form of a capsule, sachet, paper or other container. When the excipient serves as a diluent, in can be a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compounds, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The compositions of the invention can be formulated so as to provide quick, sustained or delayed release of the active ingredient after administration to the patient by employing procedures known in the art. As discussed above, given the reduced bioavailabity of ranolazine, sustained release formulations are generally preferred. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345.

The compositions are preferably formulated in a unit dosage form. The term “unit dosage forms” refers to physically discrete units suitable as unitary dosages for human subjects and other mammals, each unit containing a predetermined quantity of the active materials calculated to produce the desired therapeutic effect, in association with a suitable pharmaceutical excipient (e.g., a tablet, capsule, ampoule). The active agents of the invention are effective over a wide dosage range and are generally administered in a pharmaceutically effective amount. It will be understood, however, that the amount of each active agent actually administered will be determined by a physician, in the light of the relevant circumstances, including the condition to be treated, the chosen route of administration, the actual compounds administered and their relative activity, the age, weight, and response of the individual patient, the severity of the patient's symptoms, and the like.

For preparing solid compositions such as tablets, the principal active ingredients are mixed with a pharmaceutical excipient to form a solid preformulation composition containing a homogeneous mixture of a compound of the present invention. When referring to these preformulation compositions as homogeneous, it is meant that the active ingredients are dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage element, the latter being in the form of an envelope over the former. Ranolazine and the co-administered agent(s) can be separated by an enteric layer that serves to resist disintegration in the stomach and permit the inner element to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

Amiodarone as used in this invention is well known in the art, and is commercially available. Ranolazine may be prepared by conventional methods such as in the manner disclosed in U.S. Pat. No. 4,567,264, the entire disclosure of which is hereby incorporated by reference.

### Example 1

### Atrium-Selective Sodium Channel Block as a Novel Strategy for the Management of AF

### Background

The development of selective atrial antiarrhythmic agents is a current strategy for suppression of atrial fibrillation (AF). The present example teaches that sodium channel characteristics differ between atrial and ventricular cells and that atrium selective sodium channel block is another effective strategy for the management of AF

### Methods and Results

Whole-cell patch clamp techniques were used to evaluate inactivation of peak sodium channel current (INa) in myocytes isolated from canine atria and ventricles. The electrophysiological effects of therapeutic concentrations of ranolazine (1 to 10μmol/L) and lidocaine (2.1 to 21 μmol/L) were evaluated in canine isolated coronary-perfused atrial and ventricular preparations. The half-inactivation voltage of INa was 15 mV more negative in atrial versus ventricular cells under control conditions; this difference was increased after exposure to ranolazine. Ranolazine produced a marked use-dependent depression of sodium channel parameters, including the maximum rate of rise of the action potential upstroke, conduction velocity, and diastolic threshold of excitation, and induced postrepolarization refractoriness in atria but not in ventricles. Lidocaine also preferentially suppressed these parameters in atria versus ventricles, but to a much lesser extent than ranolazine. Ranolazine produced a prolongation of action potential duration (APD90) in atria, no effect on APD90 in ventricular myocardium, and an abbreviation of APD90 in Purkinje fibers. Lidocaine abbreviated both atrial and ventricular APD90. Ranolazine was more effective than lidocaine in terminating persistent AF and in preventing the induction of AF.

### Conclusions

Our study demonstrates important differences in the inactivation characteristics of atrial versus ventricular sodium channels and a striking atrial selectivity for the action of ranolazine to produce use-dependent block of sodium channels, leading to suppression of AF. Our results point to atrium-selective sodium channel block as a novel strategy for the management of AF.

### Example 2

### Atrial Selectivity of Ranolazine to Produce Use-Dependent Block of Sodium Channels Leading to Suppression of AF

### Background

Antiarrhythmic drug therapy remains the principal approach for suppression of atrial fibrillation (AF) and flutter (AFl) and their recurrences. Among the current strategies for suppression of AF/AFl is the development of antiarrhythmic agents that preferentially affect atrial, rather than ventricular electrical parameters. Inhibition of the ultrarapid delayed rectifier potassium current (I_{Kur}), present in atria but not in ventricles, is an example of an atrial-selective approach (Nattel et. al. Circulation. 2000;101:1179-1184.) We recently examined the hypothesis that sodium channel characteristics differ between atrial and ventricular cells and that atrial-selective sodium channel block is another effective strategy for the management of AF (Burashnikov et al. Circulation 2007;116:1449-1457 and Burashnikov et al. Heart Rhythm 2007;4:S163).

Biophysical characteristics of sodium channels were measured in single myocytes isolated from canine atria and ventricles. Four agents capable of blocking cardiac sodium channels (ranolazine, lidocaine, propafenone, and chronically administrated amiodarone) were compared with regard to their ability to alter the electro-physiology of canine coronary artery—perfused atrial and ventricular preparations as well as their ability to suppress AF. This example contrasts the effects of these open—and inactivated-state channel blockers.

### Methods and Results

Sodium Channel Inactivation Characteristics in Isolated Atrial versus Ventricular Myocytes

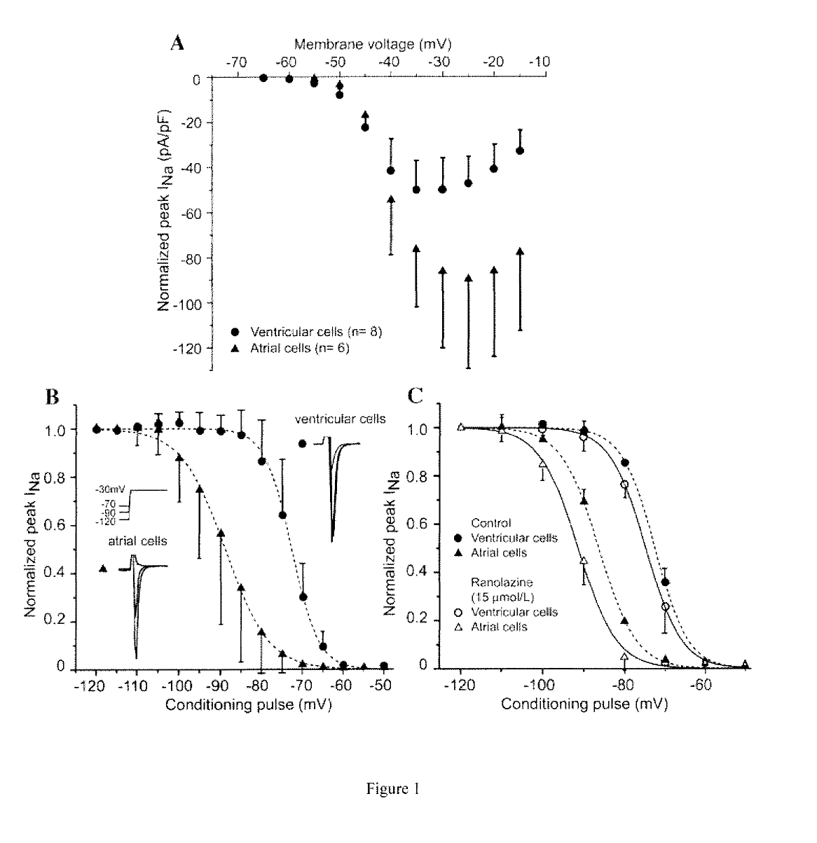

Whole-cell peak sodium currents were recorded at 37° C. in low-sodium external solution from myocytes isolated from the right atrium and left ventricle (LV) of adult mongrel dogs. The half inactivation voltage (V_{0.5}) in atrial myocytes was about 15 mV more negative than that recorded in ventricular myocytes, and the differences were increased after exposure to Ranolazine, as shown in FIG. 1.

These data indicate that a greater percentage of atrial versus ventricular sodium channels would be inactivated at a given resting or take-off potential and that recovery from sodium channel block should occur slower in atria vs. ventricles, therefore, an I_{Na} blocker could be more effective in blocking sodium channels in atria than in ventricles. An intrinsically more positive resting membrane potential (RMP) in atria (−83 mV) versus ventricles (−87 mV) would further reduce the availability of sodium channels in atria and accentuate the atrial selectivity of sodium channel blockers.

We contrasted the effects of ranolazine with other sodium channel blockers, such as lidocaine and amiodarone (predominantly inactivated state sodium channel blockers with rapid kinetics), as well as propafenone (an open-state sodium channel blocker with slow kinetics) in atria and ventricles.

### Sodium Channel-Dependent Parameters in Multicellular Atrial and Ventricular Preparations

Experiments were performed using isolated arterially perfused canine right atrial preparations and left ventricular arterially perfused wedge preparations (Antzelevitch et al. Circulation. 2004;110:904-910, Burashnikov et al. Am J Physiol. 2004;286:H2393-H2400., and Burashnikov et al. Circulation. 2003;107:2355-2360.) Therapeutic plasma concentrations of ranolazine (1-10 μM), lidocaine (2.1-21 μM), and propafenone (0.3-3.0 μM) were examined. Amiodarone was chronically administrated at a dose of 40 mg/kg/day for 6 weeks.

Sodium channel-mediated parameters, such as the maximum rate of rise of the AP upstroke (Vₘₐₓ), conduction velocity (CV), diastolic threshold of excitation (DTE), and post-repolarization refractoriness (PRR) were evaluated. PRR was defined as the difference between action potential duration (APD) and atrial effective refractory period (ERP). ERP normally coincides with APD₇₀₋₉₀, but may extend well beyond APD₇₀₋₉₀ or even APD₁₀₀ (causing the appearance of PRR) under conditions associated with a reduction of excitability (ischemia, sodium channel block, etc., see Davidenko et al. Circ Res. 1986;58:257-268).

Ranolazine and propafenone prolong APD₉₀ selectively in atria (by 11% and 13%, respectively), with little change of APD90 in the ventricles (+2% and +3%, respectively; CL=500 ms). Chronic amiodarone produced a greater prolongation of APD₉₀ in atria than in ventricles (22 versus 12%, respectively; CL=500 ms). In contrast, lidocaine abbreviates APD₉₀ in both the atria and ventricles (6% and 9%, respectively; CL=500 ms). Ranolazine, lidocaine, and chronic amiodarone lengthened ERP selectively (ranolazine) or predominantly (amiodarone and lidocaine) in atria in a rate-dependent manner, leading to the development of greater PRR in atria versus ventricles. In contrast, propafenone induced prominent PRR in both the atria and ventricles, as show in Table 1 below:

TABLE 1
The effect of ranolazine, lidocaine, propafenone, and chronic
amiodarone on sodium channel-dependent parameters in
canine isolated coronary artery-perfused atrial and ventricular
preparations

| | | Ranolazine | Lidocaine | Propafenone | Chronic |
|---|---|---|---|---|---|
| | | (10 μM) | (21 μM) | (1.5 μM) | Amiodarone |
| | | 0.5/0.3 s | 0.5/03 s | 0.5/0.3 s | 0.5/0.3 s |
| Vₘₐₓ | Atria | −26/43 | −31/40 | −46/78 | −42/67 |
| (% Δ) | Ventricles | −9/15 | −16/23 | −40/51 | −9/16 |
| DTE | Atria | +18/139 | +30/105 | +112/172 | +109/148 |
| (% Δ) | Ventricles | +3/8 | +8/40 | +84/125 | NA |
| CV | Atria | −14/46 | −29/57 | −55/97 | +25/56 |
| (% Δ) | Ventricles | −5/11 | −12/36 | −44/71 | +6/21 |
| PRR | Atria | 51/79 | 71/84 | 68/94 | 48/107 |
| (ms) | Ventricles | 3/7 | 47/69 | 52/83 | 31/36 |

Note:
Data recorded at pacing cycle lengths of 0.5 and 0.3 s.
Vₘₐₓ = maximum rate of rise of the action potential upstroke;
DTE = diastolic threshold of excitation;
CV = conduction velocity;
PRR = post-repolarization refractoriness.
PRR was determined as the difference between ERP and APD₇₅ in atria and APD₉₀ in ventricles. (ERP is coincident with APD₇₅ in atria and APD₉₀ in ventricles.)
CV was approximated from the duration of the P wave complex in atria and QRS complex in ventricles on the pseudo-ECG recordings.
n = 3-18.

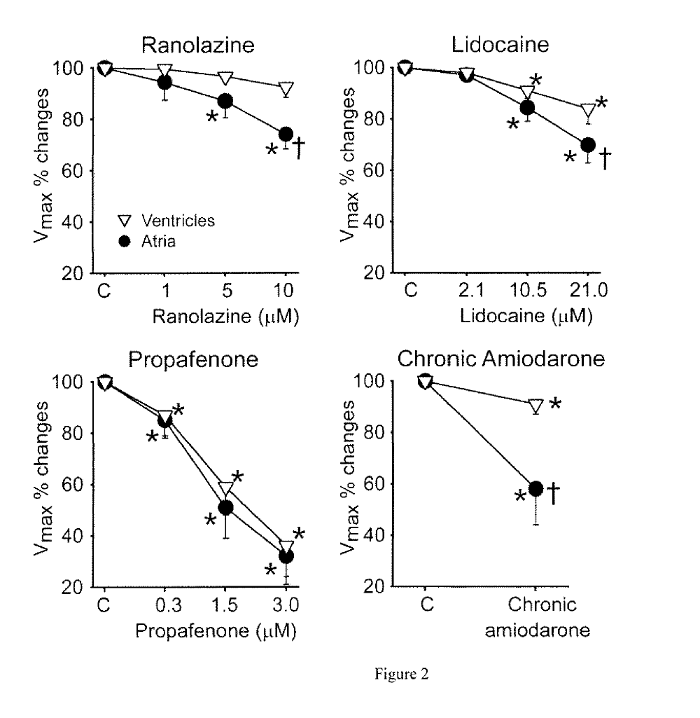

Ranolazine and chronic amiodarone caused a much greater rate-dependent reduction in Vₘₐₓ, increase in DTE, and slowing of CV in atrial than ventricular preparations as shown in FIG. 2 and Table 1. Lidocaine also preferentially suppressed these parameters in atria, although to a lesser extent. Propafenone depressed sodium channel-mediated parameters more potently than ranolazine, lidocaine, or chronic amiodarone, but without a sizable chamber selectivity at normal pacing rates (CL=500 ms). At a pacing CL of 300 ms, propafenone produced a potent depression of I_{Na}-mediated parameters in both atria and ventricles, but the effect in atria was more pronounced. This atrial selectivity of propafenone at rapid activation rates was associated with atrial-selective prolongation of APD₉₀, leading to elimination of diastolic intervals in atria but not in ventricles.

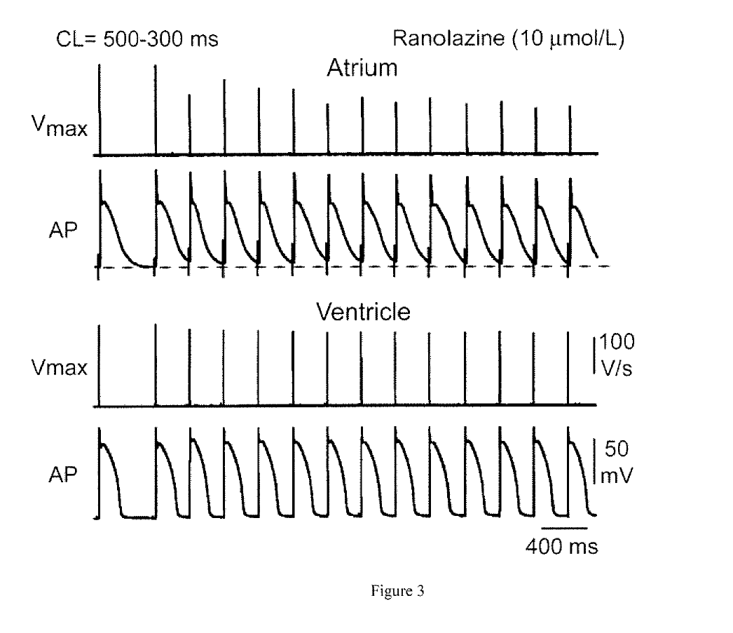

Atrial selectivity of ranolazine and amiodarone to depress I_{Na}-dependent parameters derives in part from the agents' ability to prolong APD and induce post-repolarization refractoriness predominantly in atria (due to I_{Kr} inhibition (Burashnikov et al. Heart Rhythm 2008;5:1735-1742) and thus leads to more positive take-off potential and elimination of the diastolic interval at rapid rates of activation, see FIG. 3, potentiating the actions of the drug to depress I_{Na}.

### Antiarrhythmic Effects of Ranolazine, Lidocaine, Propafenone, and Chronic Amiodarone in a Model of AF

Persistent AF is induced in 100% of canine coronary arterially perfused atrial preparations in the presence of acetylcholine (0.5 μM), see Burashnikov et al. Circulation. 2003;107:2355-2360 and Burashnikov et al. J Cardiovasc Electrophysiol. 2005;16:639-645. As shown in Table 2 below, Ranolazine was found to be more effective than lidocaine, but less effective than propafenone, in terminating acetylcholine-mediated persistent AF in coronary-perfused atria as well as in preventing the initiation of AF.

TABLE 2
Effectiveness of ranolazine, lidocaine, propafenone, and
chronic amiodarone in terminating and preventing induction
of acetylcholine-mediated AF in coronary artery-perfused
right atrial preparations

| | Ranolazine | Lidocaine | Propafenone | Chronic |
|---|---|---|---|---|
| | (10.0 μM) | (21.0 μM) | (1.5-3.0 μM) | Amiodarone |
| Termination of | 66% (4/6) | 33% (2/6) | 100% (7/7) | NA |
| AF | | | | |
| Prevention of | 80% (8/10) | 57% (4/7) | 100% (6/6) | 83% (5/6) |
| induction of AF | | | | |

Note:
Persistent AF was inducible in 100% of atria in the presence of acetylcholine alone.

Persistent acetylcholine-mediated AF could be induced in only 1 of 6 atria isolated from dogs chronically treated with amiodarone (versus 10 of 10 untreated atria). Anti-AF actions of ranolazine, lidocaine, propafenone, and amiodarone were associated with the development of significant rate-dependent PRR.

Ranolazine (5-10 μM) also prevented the induction of AF in 4 of 5 atria in which self-terminating AF was induced by exposure to ischemia and β-adrenergic agonists (Burashnikov et al. Circulation. 2007;116:1449-1457 and Burashnikov et al. [abstract]. Heart Rhythm. 2005;2:S179). Ischemia/reperfusion coupled with iso-proterenol mimics the conditions that prevail during acute myocardial infarction or the substrate encountered postsurgically.

### Discussion

Our recent studies demonstrate very significant differences in the inactivation characteristics of atrial versus ventricular sodium channels and a striking atrial selectivity for the action of ranolazine to produce use-dependent block of the sodium channels, leading to depression of excitability, development of PRR, and suppression of AF (Burashnikov et al. Circulation. 2007;116:1449-1457 and Burashnikov et al. [abstract]. Heart Rhythm. 2007;4:S163). Lidocaine and chronic amiodarone also depressed sodium channel-dependent parameters (Vₘₐₓ, CV, DTE, and PRR) predominantly in atria. It is noteworthy that lidocaine was much less atrial-selective than ranolazine or chronic amiodarone (see Table 1 above). In contrast, propafenone is not atrial-selective.

Available data suggest that open vs. inactivated state block of the sodium channel does not determine the potential for atrial selectivity. While both propafenone and ranolazine are predominantly open state blockers amiodarone and lidocaine are predominantly inactivated state blockers (Wang et al. Mol Pharmacol 2008;73:940-948 and Zygmunt et al. Biophys J;2009:96:250a [abstract], Burashnikov et al. Circ. 2007;116:1449-1457, Kodama et al. Cardiovasc Res 1997;35:13-29; Whalley et al PACE 1995;18:1686-1704). Rate of dissociation of drug from the sodium channel on the other hand is thought to contribute to atrial selectivity. Ranolazine and amiodarone, both atrial-selective sodium channel blockers, possess relatively rapid dissociation kinetics (unbinding τ=0.2-1.6 sec) (Kodama et al. Cardiovasc Res 1997;35:13-29; Burashnikov et al. Circ. 2007;116:1449-1457) whereas propafenone, which shows little to no atrial selectivity, displays slow dissociation kinetics (unbinding τ≧8 sec) (Whalley et al PACE 1995;18:1686-1704). Validation of this hypothesis awaits assessment of the atrial selectivity of other “slow” I_{Na}.

In canine ventricular myocytes, ranolazine has been shown to inhibit late I_{Na} with an IC₅₀ of 6 μM, see Antzelevitch et al. Circulation. 2004;110:904-910, but to inhibit peak I_{Na} with an IC₅₀ of 294 μM, see Undrovinas et al. J Cardiovasc Electrophysiol. 2006;17:S161-S177. Consistent with the latter, ranolazine has been reported to suppresses Vₘₐₓ with an IC₅₀ of >100 μM in ventricular Purkinje fibers and M ceil preparations paced at a CL of 500 ms (Antzelevitch et al. Circulation. 2004;110:904-910 and Antzelevitch et al. J Cardiovasc Pharmacol Therapeut. 2004;9(Suppl 1):S65-S83). In sharp contrast, ranolazine causes a prominent use-dependent reduction of I_{Na} (estimated based on changes in Vₘₐₓ) in atrial preparations at concentrations within the therapeutic range of ranolazine (2-10 μM), see Burashnikov et al. Circulation. 2007;116:1449-1457.

Sodium channel blockers generally bind more effectively to open and/or inactivated sodium channels (i.e., during the action potential) than to resting sodium channels (i.e., during the diastolic interval). Unblocking occurs largely during the resting state (Whalley et al. Pacing Clin Elecrophysiol. 1995;18:1686-1704). Rapid activation rates contribute to the development of sodium channel block by increasing the proportion of time that the sodium channels are in the open/inactivated state and reducing the time that the channels are in the resting state. As shown in FIG. 3, agents that prolong APD selectively in atria but not ventricles are expected to display atrial-selective I_{Na} block, particularly at rapid activation rates on account of their ability to reduce or eliminate the diastolic interval and depolarize take-off potential in an atrial-selective manner. The more depolarized RMP in atria potentiates the effects of I_{Na} blockers by increasing the fraction of channels in the inactivated state, which reduces the availability of sodium channels and prolongs the time needed for the sodium channels to recover from inactivation.

Ranolazine was more atrial-selective than was lidocaine and more effective than lidocaine in terminating and preventing recurrence of AF. This may be due to the fact that ranolazine prolongs only atrial APD because of its ability to also block the rapidly activating delayed rectifier potassium current (I_{Kr}, IC₅₀=12 μM), (Antzelevitch et al. Circulation. 2004;110:904-910) whereas lidocaine, a more selective I_{Na} blocker, abbreviates both atrial and ventricular APD. It is noteworthy that I_{Kr} blockers preferentially prolong atrial versus ventricular APD (see below). The selective prolongation of APD in atria by ranolazine leads to elimination of diastolic intervals and more depolarized take-off potentials at rapid rates in atria but not ventricles, also shown in FIG. 3. The more negative h-curve in atria and acceleration-induced depolarization of take-off potential act in concert to increase the fraction of channels in the inactivated state, making sodium channels less available and more sensitive to block by ranolazine. The result is a greater atrial versus ventricular suppression of I_{Na}-dependent parameters such as Vₘₐₓ, DTE, and CV, and the development of use-dependent PRR.

The effect of ranolazine to prolong atrial repolarization potentiates but does not appear to be a determining factor in ranolazine's atrial specificity and in antiarrhythmic efficacy. Propafenone (I_{Na} and I_{Kr} blocker), like ranolazine, selectively prolongs atrial APD₉₀ but suppresses I_{Na}-dependent parameters in both the atrial and the ventricular preparations to a similar extent at a CL 500 ms (Burashnikov et al Circulation. 2007;116:1449-1457), as does GE 68, a propafenone analogue, see Lemmens-Gruber et al. Arch Pharmacol. 1997;355:230-238. At faster pacing rates, propafenone more effectively depresses Vₘₐₓ and CV in atria on account of atrial-selective APD₉₀ prolongation (leading to elimination of diastolic interval in atria). Lidocaine abbreviates both atrial and ventricular APD₉₀, but shows atrial specificity in depression of I_{Na}-dependent parameters. Chronic amiodarone produces depression of I_{Na}-dependent parameters pre-dominantly in atria via a similar mechanism, which includes preferential prolongation of atrial APD.

These results suggest that the I_{Kr} blocking effect of ranolazine, chronic amiodarone, and propafenone potentiates sodium channel inhibitory effect of these drugs in atria at fast pacing rates. Interestingly, I_{Kr} blockers generally produce a much greater APD prolongation in atria than in ventricles (Burashnikov et al. Heart Rhythm 2008;5:1735-1742). Selective inhibition of I_{Kr} prolongs atrial ERP more than ventricular ERP at normal or moderately rapid activation rates, (Spinelli et al. J Cardiovasc Pharmacol. 1992;20:913-922 and Wiesfeld et al. J Cardiovasc Pharmacol. 1996;27:594-600) but not at slow rates. At relatively slow activation rates or following long pauses, I_{Kr} block preferentially prolongs ventricular versus atrial APD, leading to development of early afterdepolarization (EAD) and torsade de pointes arrhythmias in the ventricles, but not in atria, see Antzelevitch et al. J Cardiovasc Electrophysiol. 1999;10:1124-1152, Burashnikov et al. Pacing Clin Electrophysiol. 2006;29:290-295, and Vincent et al. J Cardiovasc Electrophysiol. 2003;14:1034-1035).

A number of antiarrhythmic agents have been shown to be effective in terminating and/or preventing clinical AF/AFl. Most of these agents have as a primary action the ability to reduce I_{Na} (e.g., propafenone or flecainide) and I_{Kr} (e.g., dofetilide) or to inhibit multiple ion channels, as in the case of amiodarone. An important limitation of these antiarrhythmic agents is their potential ventricular proarrhythmic actions and/or organ toxicity at therapeutically effective doses (Antzelevitch et al. J Cardiovasc Pharmacol Therapeut. 2004;9(Suppl 1):S65-S83, Antzelevitch et al. J Cardiovasc Electrophysiol. 1999;10:1124-1152, and Cardiac Arrhythmia Suppression Trial (CAST) Investigators. Preliminary report: effect of encainide and flecainide on mortality in a randomized trial of arrhythmia suppression after myocardial infarction. N Engl J Med. 1989;321:406-412).

This has prompted the development of atrial-selective antiarrhythmic agents, such as those that block I_{Kur} (Nattel et al. Circulation. 2000;101:1179-1184, Wang et al. Circ Res. 1993;73:1061-1076, and Amos et al. J Physiol. 1996;491(Pt 1):31-50). However, block of I_{Kur} alone may not be sufficient for the suppression of AF (Burashnikov et al. Heart Rhythm. 2008;5:1304-1309; Burashnikov et al Expert Opinion Emerging Drugs 2009;14(2):233-249). In remodelled atria, I_{Kur} block selectively prolongs atrial APD₉₀ (but only slightly) and, when combined with Iₜₒ (perhaps with I_{K-ACh}) and/or I_{Na} inhibition, can suppress AF/AFl (Burashnikov et al. Heart Rhythm. 2008;5:1304-1309 and Blaauw et al. Circulation. 2004;110:1717-1724). In non-remodelled healthy atria, I_{Kur} inhibition abbreviates APD₉₀, (Burashnikov et al. Am J Physiol. 2004;286:H2393-H2400, Burashnikov et al. Heart Rhythm. 2008;5:1304-1309, and, Wettwer et al. Circulation, 2004;110:2299-2306) and can promote AF (Burashnikov et al. Heart Rhythm. 2008;5:1304-1309 ).

These data are consistent with the results of a recent study showing an association of loss-of-function mutations in KCNA5, which encodes the α-subunit of I_{Kur} channel, with familial AF, see Olson et al. Hum Mol Genet. 2006;15:2185-2191. Our results suggest that atrial-selective sodium channel block may be another effective approach for the management of AF.

### Conclusions

Our findings suggest that sodium channel blockers with relatively rapid kinetics for block of the sodium channel have a proclivity towards producing atrial-selective sodium channel inhibition. The results point to atrial-selective sodium channel block as a novel strategy for the management of AF and suggest that the additional presence of I_{Kr} block and APD prolongation can potentiate the atrial selectivity of I_{Na} blockers and thus enhance their effectiveness in suppressing and preventing the development of AF.

### Example 3

### Combined Treatment with Ranolazine and Amiodarone

Ranolazine (RAN) is an antianginal agent recently shown to possess antiarrhythmic activity in ventricular and atrial myocytes, including pulmonary vein (PV) sleeve preparations. Chronic amiodarone (Amio) is commonly used for the treatment of supraventricular and ventricular arrhythmias, including atrial fibrillation (AF). Delayed afterdepolarizations (DADs) and late phase 3 early afterdepolarizations (EADs), originating from PV sleeves, have been proposed as potential triggers in the initiation of AF. This study was designed to evaluate the electrophysiologic and antiarrhythmic effects of ranolazine in superfused PV sleeve preparations isolated from dogs treated with chronic Amio (6 weeks, 40 mg/kg daily).

### Methods:

Action potentials (AP) were recorded from canine superfused PV sleeves using microelectrode techniques. Acetylcholine (ACh, 1 μM), isoproterenol (Iso, 1 μM), or their combination was used to induce EADs, DADs, and triggered activity.

### Results:

PV sleeves isolated from dogs treated with chronic Amio exhibited a much lower maximal rate of rise of AP upstroke (Vmax) and a prolonged AP duration compared to control (untreated) PV sleeve preparations; Vmax was 314±79 V/s in untreated controls and 115±89 V/s in chronic Amio PV sleeves at a cycle length (CL) of 1000 ms. 2:1 activation failure developed at an average CL of 420 ms (vs. 124 ms in PV preparations isolated from untreated dogs). Superfusion with RAN (5 and 10 μM, n=5) further depressed excitability of PV sleeves leading to 2:1 activation failure at an average CL of 1350 and 1660 ms, after addition of 5 and 10 μM RAN respectively. In untreated controls, 2:1 activation failure occurred at 190 ms in the presence of 10 μM ranolazine. In chronic Amio PV sleeves, late phase 3 EAD- and DAD-induced triggered activity were rarely observed in the presence of Iso and/or ACh following rapid pacing and, when observed, were completely eliminated by the addition of RAN (5-10 μM).

### Conclusions:

RAN added to chronic Amio-treated PV sleeve preparations greatly potentiates the effects of chronic Amio to depress excitability, leading to activation failure at relatively long CLs and complete suppression of triggered activity. The combined effect of chronic Amio and acute RAN suggest that RAN may help suppress AF in patients in whom Amio was not effective. The synergistic effect of the combination can lead to a “pharmacologic” ablation of the pulmonary veins.

Example 4

### Synergistic Effect of Ranolazine and Amiodarone

Recent studies by us and others have demonstrated that chronic amiodarone is an atrial-selective inactivated-state blocker of cardiac sodium channels and that ranolazine is an atrial-selective activated-state blocker of these channels. We hypothesized that the combination would act synergistically to cause use-dependent depression of sodium channel activity in the atrium.

### Methods:

The electrophysiological as well as antiarrhythmic effects of acute ranolazine (5 μM) were studied in arterially-perfused right atrial preparations isolated from untreated (n=7) and chronic AMIO treated (n=4; 40 mg/kg daily for 6 weeks) dogs. Floating microelectrode techniques were used to record transmembrane action potentials (BCL=500 ms).

### Results:

Action potential duration of pectinate muscle (PM-APD₇₅) and effective refractory period (ERP) were significantly longer in Amio vs. untreated atria (APD: 183±7 vs. 154±11 ms; ERP: 222±12 vs. 158±18 ms; p<0.05 for each). Ranolazine slightly prolonged APD₇₅ in AMIO and untreated controls (from 183±7 to 189±9 ms and from 154±11 to 159±9 ms, respectively, both p=n.s.), but significantly prolonged ERP particularly in AMIO (from 189±9 to 258±50 ms, p<0.01) vs. untreated atria (from 158±18 to 190±24 ms, p<0.05). Thus, ERP prolongation was largely due to the development post-repolarization refractoriness (PRR).

The shortest pacing CL permitting a 1:1 response was 129±8 in control, 221±39 with AMIO, 234±49 after acute ranolazine and 325±34 ms after the combination of AMIO and ranolazine (p<0.01 vs. either drug alone) reflecting reduced excitability and accentuated PRR. In presence of acetylcholine (ACh), the shortest pacing CL permitting 1:1 response was 71±12 in control, 136±22 with chronic AMIO, 94±31 with acute ranolazine, and 205±34 ms with AMIO+ranolazine. In ACh-pretreated preparations, burst pacing induced atrial fibrillation in 100% of controls (10/10) but in 0% of preparations treated with AMIO and ranolazine.

### Conclusions:

The combination of chronic amiodarone and relatively low concentrations of acute ranolazine produces a synergistic use-dependent depression of sodium channel-dependent parameters in isolated canine atria, leading to a potent effect of the drug combination, to prevent the induction of AF.

We claim:

1. A method of treating atrial fibrillation comprising the coadministration of a synergistic therapeutically effective amount of amiodarone and synergistic therapeutically effective amount ranolazine.

2. The method of claim 1, wherein the ranolazine are administered orally.

3. The method of claim 1, wherein the amiodarone and ranolazine are administered separately.

4. The method of claim 1, wherein the ranolazine and amiodarone are administered intravenously.

5. The method of claim 2, wherein the amiodarone and ranolazine are administered as a combined dosage unit.

6. The method of claim 2, wherein the amount of ranolazine administered is 3000 mg daily, 1500 mg daily, 1000 mg daily, or 750 mg daily.

7. The method of claim 6, wherein the ranolazine is administered as a sustained release formulation.

8. The method of claim 2, wherein the amount of amiodarone administered is as a loading dose of 1200-1600 mg daily for 7-10 days, followed by a maintenance dose of 200, 100 or 50 mg daily.

9. The method of claim 4, wherein the amount of amiodarone administered is as a loading dose of 15 mg/min for the first 10 minutes followed by 1 mg/min for the next two to four hours, followed by a maintenance dose of 540 mg, 360 mg, or 180 mg per day.

10. A method for reducing the undesirable side effects of amiodarone comprising coadministration of a synergistic therapeutically effective amount ranolazine.

11. The method of claim 10, wherein the amiodarone and ranolazine are administered orally.

12. The method of claim 10, wherein the amiodarone and ranolazine are administered separately.

13. The method of claim 10, wherein the ranolazine and amiodarone are administered intravenously.

14. The method of claim 10, wherein the amiodarone and ranolazine are administered as a combined dosage unit.

15. The method of claim 11, wherein the amount of ranolazine administered is 3000 mg daily, 1500 mg daily, 1000 mg daily, or 750 mg daily.

16. The method of claim 11, wherein the ranolazine is administered as a sustained release formulation.

17. The method of claim 11, wherein the amount of amiodarone administered is 200, 100 or 50 mg daily

18. A pharmaceutical formulation comprising a synergistic therapeutically effective amount of amiodarone and synergistic therapeutically effective amount ranolazine.

19. A method for reducing the therapeutically effective dose of amiodarone comprising coadministration of a therapeutically effective amount ranolazine.

20. The method of claim 19, wherein the ranolazine is administered orally.

21. The method of claim 19, wherein the amiodarone and ranolazine are administered separately.

22. The method of claim 19, wherein the ranolazine and amiodarone are administered intravenously.

23. The method of claim 19, wherein the amiodarone and ranolazine are administered as a combined dosage unit.

24. The method of claim 20, wherein the amount of ranolazine administered is 3000 mg daily, 1500 mg daily, 1000 mg daily, or 750 mg daily.

25. The method of claim 20, wherein the ranolazine is administered as a sustained release formulation.

26. The method of claim 19, wherein the amount of amiodarone administered is reduced to 200, 100 or 50 mg daily.
